(19) United States
(12) Patent Application Publication
(10) Pub. No.: US 2022/0071993 A1
(43) Pub. Date: Mar. 10, 2022

(54) A VILAZODONE SOLID DISPERSION AND PREPARATION METHOD THEREOF

(71) Applicant: SUNSHINE LAKE PHARMA CO., LTD., Dongguan, Guangdong (CN)

(72) Inventors: Dailong FANG, Dongguan (CN); Lulu FAN, Dongguan (CN); Xin HUANG, Dongguan (CN); Jinsong YOU, Dongguan (CN); Fangfang HUANG, Dongguan (CN)

(73) Assignee: SUNSHINE LAKE PHARMA CO., LTD., Dongguan, Guangdong (CN)

(21) Appl. No.: 17/311,784

(22) PCT Filed: Dec. 11, 2019
(86) PCT No.: PCT/CN2019/124429

§ 371 Date: Nov. 26, 2021

(30) Foreign Application Priority Data
Dec. 13, 2018 (CN) 201811523043.2

(51) Int. Cl. A61K 31/496; A61K 47/34; A61K 47/32; A61K 47/38

(52) U.S. Cl. CPC A61K 31/496; A61K 47/38; A61K 47/32; A61K 47/34

(57) ABSTRACT

Provided are a vilazodone solid dispersion, a preparation method therefor and a use thereof, wherein the vilazodone solid dispersion contains an active ingredient' vilazodone, a water-soluble polymer carrier material and a surfactant. The water-soluble polymer carrier material is selected from at least one of polyvidone, copovidone and hydroxypropyl methylcellulose. The vilazodone is present in the solid dispersion in a non-crystalline state. The solid dispersion has good stability and significantly improves the solubility and in-vitro dissolution rate of the vilazodone, thereby significantly increasing the oral bioavailability thereof. The vilazodone solid dispersion can be used for preparing vilazodone-related preparations.

### FIELD OF THE INVENTION

This application claims the priority and benefits of Chinese Patent Application No. 201811523043.2, filed with the State Intellectual Property Office of China on Dec. 13, 2018, which is incorporated herein by reference in its entirety.

The invention relates to the field of biomedicine, in particular to a vilazodone solid dispersion and preparation method and application thereof.

### BACKGROUND OF THE INVENTION

Vilazodone is the first indole alkylamine antidepressant, belongs to selective serotonin reuptake inhibitor and 5-HT1A receptor partial agonist. The U.S. Food and Drug Administration (FDA) approved vilazodone hydrochloride tablet for the treatment of moderate to severe depression on Jan. 21, 2011. The trade name of its commercially formulation is VIIBRYD®, strength contains 10 mg, 20 mg, and 40 mg, respectively. The recommended dose for clinical medication is 40 mg/once/day, adopting a gradual escalation dosing regimen. The initial dose is 10 mg/once/day and the administration is continued for 7 days, then the dose is 20 mg/once/day and the administration is continued for 7 days, and the final dose is maintained at 40 mg/once/day.

After oral administration of VIIBRYD® tablet, it is mainly absorbed in the small intestine, and its plasma drug concentration peak time (Tmax) is 4 to 5 hours. However, due to the low solubility of vilazodone hydrochloride, it has poor solubility in gastric juice (pH 1.2) and intestinal juice (pH 4.5 to 6.8), and only has a certain solubility in an environment with pH 3.1. Thus its fasting oral bioavailability is low, leading to extremely low bioavailability of VIIBRYD® when taken on an empty stomach, which is difficult to meet clinical treatment needs. Therefore, the instruction of VIIBRYD® clearly indicates that the drug must be taken with food; under feeding condition, the pH of human gastric juice is about 3.1, and it can delay the gastric emptying of the drug, ultimately leading to that the oral bioavailability of VIIBRYD® is about 72% under feeding condition, and under fasting condition, its AUC (blood drug concentration) and Cₘₐₓ (maximum peak concentration) decrease by 50% and 60%, respectively.

In order to improve the solubility of vilazodone hydrochloride, the prior art discloses that preparing it into an amorphous state can increase its solubility.

Patent application IN2012CH03815A discloses a solid dispersion prepared by vilazodone hydrochloride and copovidone (S-630) or povidone (PVP-K30). The X-ray diffraction (XRD) pattern shows that the vilazodone hydrochloride is amorphous existing in the carrier material, but the literature does not give the solubility or in vitro dissolution rate data of the prepared vilazodone hydrochloride solid dispersion; patent application US20140323498 discloses an amorphous solid dispersion prepared by vilazodone hydrochloride and povidone (PVP) or hydroxypropyl methylcellulose (HPMC); patent application US20130324554 discloses an amorphous solid dispersion prepared by vilazodone hydrochloride and povidone (PVP), hydroxypropyl cellulose (HPC) or hydroxypropyl methylcellulose phthalate (HPMCP); patent application US20150126525 discloses an amorphous solid dispersion prepared by vilazodone hydrochloride and povidone (PVP) or hydroxypropyl methylcellulose (HPMC); patent application IN2013MU03458 discloses an amorphous solid dispersion prepared by vilazodone hydrochloride and copovidone or Eudragit material; patent application CN106580895 discloses an amorphous solid dispersion prepared by vilazodone hydrochloride and povidone (PVP) or polyethylene glycol (PEG); patent application CN106580914 discloses an amorphous solid dispersion prepared by vilazodone hydrochloride and polyethylene glycol (PEG) or acrylic resin. However, none of the above-mentioned prior art literatures disclose the solubility or in vitro dissolution rate data of the prepared solid dispersion. Whether the prepared solid dispersion can increase the solubility of vilazodone hydrochloride and the oral bioavailability under fasting condition is not clear.

Patent application CN104983711 discloses a solid dispersion prepared by vilazodone hydrochloride and povidone (PVP) or polyethylene glycol (PEG) at a weight ratio of 1:2.5. The dissolution rate of the prepared solid dispersion is improved under low strength (10 mg) condition comparing with the ordinary formulation, but the degree of improvement is limited, and the highest dissolution platform is about 90%. Theoretically, the dissolution rate is lower under high strength (40 mg) condition, and the oral bioavailability is limited under fasting condition, which cannot meet the needs of clinical medication. In addition, due to the proportion of carrier material in the formulation is too low (1:2.5), the stability of the later formulation may have a problem, which may eventually lead to low oral bioavailability.

As we all know, when using solid dispersion technology to improve the solubility and oral bioavailability of poorly soluble drugs, there are two important problems: (1) under high strength condition, the dissolved drugs are easy to re-crystallize, resulting in a decrease in dissolution rate and ultimately a decrease in bioavailability; (2) during storage, the solid dispersion is prone to aging and crystallization occurs, resulting in a decrease in dissolution rate and bioavailability.

Therefore, in order to meet the needs of clinical medication, it is urgent to provide a solid dispersion of vilazodone hydrochloride with better stability and higher solubility for the treatment of moderate to severe depression.

### SUMMARY OF THE INVENTION

In order to solve the problem of low fasting bioavailability caused by the low solubility of vilazodone, the present invention provides a vilazodone solid dispersion with better stability and higher solubility, the solid dispersion comprises vilazodone, carrier material and surfactant, the carrier material is a water-soluble polymer carrier material. Unless otherwise specified, the vilazodone herein may be vilazodone free base, or a pharmaceutically acceptable salt or ester thereof, specifically 5-{4-[4-(5-cyano-3-indole)-butyl]-1-piperazinyl}-coumarone-2-carboxamide or pharmaceutically acceptable salt or ester thereof.

According to common knowledge, when preparing solid dispersion, the solubilization of active pharmaceutical ingredient (API) varies greatly using different types and proportions of carrier materials. Therefore, it is not easy to obtain a formulation with strong solubilization and good stability. After the inventors studied, the invention provides a vilazodone solid dispersion, which has better solubilization and better stability.

In the first aspect of the invention, a vilazodone solid dispersion is proposed, the solid dispersion comprises vilazodone, a carrier material and a surfactant.

According to the embodiments of the present invention, the carrier material is a water-soluble polymer carrier material, and the carrier material includes at least one selected from povidone, copovidone, and hydroxypropyl methylcellulose. Through the research on the carrier material, a vilazodone solid dispersion is prepared by using povidone, copovidone, or hydroxypropyl methylcellulose as a carrier material. The dissolution rate of the vilazodone solid dispersion in a simulated small intestine environment (pH 6.8 medium) is greater than 50%, significantly higher than that of commercially formulation, which can significantly improve the oral bioavailability of vilazodone on an empty stomach.

According to the embodiments of the present invention, on the basis of the carrier material, a surfactant is added, and the surfactant has a greater influence on the improvement of the dissolution rate and the improvement of the stability of the obtained solid dispersion.

According to the embodiments of the present invention, the carrier material is a water-soluble polymer carrier material. By investigating the ratio of vilazodone to water-soluble polymer carrier material, it is found that, when the weight ratio of vilazodone to water-soluble polymer carrier material is less than or equal to 1:4, vilazodone exists in an amorphous form in the solid dispersion, and its dissolution rate in vitro is significantly higher than that of commercially formulation. In addition, considering the toxicity problems caused by a large number of carrier materials and the difficulties in the preparation of later formulations, the weight ratio of vilazodone to carrier material in the solid dispersion is 1:4 to 1:7 in the present invention. In some embodiments, the weight ratio of vilazodone to carrier material is 1:4 to 1:5; in some embodiments, the weight ratio of vilazodone to carrier material is 1:4 to 1:6; in some embodiments, the weight ratio of vilazodone to carrier material is 1:5 to 1:7; in some embodiments, the weight ratio of vilazodone to carrier material is 1:5 to 1:6; in some embodiments, the weight ratio of vilazodone to carrier material is 1:6 to 1:7. In some embodiments, the weight ratio of vilazodone to carrier material is 1:4; in some embodiments, the weight ratio of vilazodone to carrier material is 1:5; in some embodiments, the weight ratio of vilazodone to carrier material is 1:6; in some embodiments, the weight ratio of vilazodone to carrier material is 1:7.

According to the embodiments of the present invention, the vilazodone solid dispersion further comprises a surfactant, and the surfactant includes but is not limited to the following categories:

Polyoxyethylene alkyl ethers, such as polyoxyethylene (3) lauryl ether, polyoxyethylene (5) cetyl ether, polyoxyethylene (2) stearyl ether, polyoxyethylene (5) stearyl ether; polyoxyethylene alkyl aryl ethers, such as polyoxyethylene (2) nonyl phenyl ether, polyoxyethylene (3) nonyl phenyl ether, polyoxyethylene (4) nonyl phenyl ether, polyoxyethylene (3) octyl phenyl ether; polyethylene glycol glycerides, such as PEG-200 monolaurate, PEG-200 dilaurate, PEG-300 dilaurate, PEG-400 dilaurate, PEG-300 distearate, PEG-300 dioleate; alkylene glycol fatty acid monoesters, such as propylene glycol monolaurate; sucrose fatty acid esters, such as sucrose monostearate, sucrose distearate ester, sucrose monolaurate, sucrose dilaurate; sorbitan fatty acid monoesters, such as sorbitan monolaurate, sorbitan monooleate, sorbitan monopalmitate, sorbitan boronic acid ester, or one or more of the above mixtures.

Wherein, the HLB (hydrophilic-lipophilic balance) value of the surfactant is not less than 6; in some embodiments, the surfactant includes at least one selected from tween, Span-20, polyoxyethylene castor oil, polyoxyethylene 40 hydrogenated castor oil (Cremophor RH40), caprylic acid capric acid polyethylene glycol glyceride (Labrasol), polyethylene glycol hydroxystearate, lauric acid polyethylene glycol glyceride, poloxamer, (D)α-tocopherol polyethylene glycol succinate and sodium lauryl sulfate.

According to the embodiments of the present invention, the weight ratio of the surfactant in the vilazodone solid dispersion is not more than 20%, which is beneficial to the solubilization and/or stability of the vilazodone solid dispersion. In some embodiments, the weight ratio of the surfactant in the vilazodone solid dispersion is not more than 10%, which is beneficial to the solubilization and/or stability of the vilazodone solid dispersion. In some embodiments, the weight ratio of the surfactant in the vilazodone solid dispersion is 20%. In some embodiments, the weight ratio of the surfactant in the vilazodone solid dispersion is 10%.

According to the embodiments of the present invention, the vilazodone solid dispersion comprises vilazodone, a carrier material and a surfactant, the carrier material may be copovidone. The vilazodone solid dispersion prepared by using copovidone as a carrier material has a large dissolution rate in a simulated small intestine environment (pH 6.8 medium), and can significantly improve the oral bioavailability of vilazodone on an empty stomach. According to the embodiments of the present invention, the weight ratio of vilazodone to copovidone in the vilazodone solid dispersion is 1:4 to 1:7. In some embodiments, the weight ratio of the vilazodone to copovidone is 1:6.

According to the embodiments of the present invention, in the vilazodone solid dispersion, the surfactant may be poloxamer 188. According to some embodiments of the present invention, the vilazodone solid dispersion comprises vilazodone, copovidone and poloxamer 188, and the weight ratio of the poloxamer 188 in the vilazodone solid dispersion is not more than 20%. In some embodiments, the weight ratio of the poloxamer 188 in the vilazodone solid dispersion is not more than 10%. In some embodiments, the weight ratio of the poloxamer 188 in the vilazodone solid dispersion is 20%; In some embodiments, the weight ratio of the poloxamer 188 in the vilazodone solid dispersion is 10%.

According to some embodiments of the present invention, the vilazodone solid dispersion comprises vilazodone, a carrier material and a surfactant, the carrier material may be povidone. The vilazodone solid dispersion prepared by using povidone as a carrier material has a large dissolution rate in a simulated small intestine environment (pH 6.8 medium), and can significantly improve the oral bioavailability of vilazodone on an empty stomach.

According to the embodiments of the present invention, the weight ratio of vilazodone to povidone in the vilazodone solid dispersion is 1:4 to 1:7. In some embodiments, the weight ratio of vilazodone to povidone is 1:5; in some embodiments, the weight ratio of vilazodone to povidone is 1:6.

According to some embodiments of the present invention, the vilazodone solid dispersion comprises vilazodone, povidone and poloxamer 188. According to some embodiments of the present invention, the vilazodone solid dispersion comprises vilazodone, povidone and Span-20. According to some embodiments of the present invention, the vilazodone solid dispersion comprises vilazodone, povidone, poloxamer 188 and Span-20. According to some embodiments of the present invention, the weight ratio of the Span-20 in the vilazodone solid dispersion is not more than 10%; in some embodiments, the weight ratio of the Span-20 in the vilazodone solid dispersion is not more than 5%. In some embodiments, the weight ratio of the Span-20 in the vilazodone solid dispersion is 10%; in some embodiments, the weight ratio of the Span-20 in the vilazodone solid dispersion is 5%.

According to the embodiments of the present invention, the vilazodone solid dispersion comprises vilazodone, a carrier material and a surfactant, the carrier material may be hydroxypropyl methylcellulose. The vilazodone solid dispersion prepared by using hydroxypropyl methylcellulose as a carrier material has a large dissolution rate in a simulated small intestine environment (pH 6.8 medium), and can significantly improve the oral bioavailability of vilazodone on an empty stomach.

According to the embodiments of the present invention, the weight ratio of vilazodone to hydroxypropyl methylcellulose in the vilazodone solid dispersion is 1:4 to 1:7. In some embodiments, the weight ratio of vilazodone to hydroxypropyl methylcellulose is 1:6.

According to some embodiments of the present invention, the vilazodone solid dispersion comprises vilazodone, hydroxypropyl methylcellulose, and caprylic acid capric acid polyethylene glycol glyceride. According to some embodiments of the present invention, the vilazodone solid dispersion comprises vilazodone, hydroxypropyl methylcellulose, and polyoxyethylene 40 hydrogenated castor oil. According to some embodiments of the present invention, the vilazodone solid dispersion comprises vilazodone, hydroxypropyl methylcellulose, caprylic acid capric acid polyethylene glycol glyceride, and polyoxyethylene 40 hydrogenated castor oil. According to some embodiments of the present invention, the weight ratio of the caprylic acid capric acid polyethylene glycol glyceride in the vilazodone solid dispersion is not more than 10%. According to some embodiments of the present invention, the weight ratio of the polyoxyethylene 40 hydrogenated castor oil in the vilazodone solid dispersion is not more than 10%. In some embodiments, the weight ratio of the caprylic acid capric acid polyethylene glycol glyceride in the vilazodone solid dispersion is 10%. In some embodiments, the weight ratio of the polyoxyethylene 40 hydrogenated castor oil in the vilazodone solid dispersion is 10%.

The present invention investigates the addition of surfactants on the basis of a single carrier material. The results show that for different carrier materials, the types of surfactant have a greater impact on the improvement of dissolution rate and stability. When poloxamer 188 is added into the copovidone material, the dissolution rate of the prepared vilazodone solid dispersion is significantly improved and stable, after other surfactants are added, the dissolution rate is improved but unstable, and there is a decline in the later period. Similarly, when poloxamer 188 or Span 20 is added into povidone, or polyoxyethylene 40 hydrogenated castor oil (Cremophor RH40) or caprylic acid capric acid polyethylene glycol glycerin (Labrasol) is added into the hydroxypropyl methylcellulose material, the prepared vilazodone solid dispersion has high in vitro dissolution rate and stability, which is significantly higher than the dissolution rate of the commercially formulation (VIIBRYD®), and can significantly improve the oral bioavailability of vilazodone under fasting condition. The results show that on the basis of the carrier material, the addition of a specific surfactant can further increase the solubility of vilazodone hydrochloride and/or inhibit its crystallization, reduce the drop of dissolution rate, and help improve the stability of the dispersion.

According to the embodiments of the present invention, in some embodiments, the vilazodone solid dispersion comprises vilazodone and a water-soluble polymer carrier material, the water-soluble polymer carrier material is a mixed material, the mixed material comprises at least two of povidone, copovidone, and hydroxypropyl methylcellulose. In some embodiments, the present invention has found that when the dissolution rate is higher (greater than 70%), the dissolution rate of the solid dispersion (without surfactant) prepared by using different ratios of a single material has decreased to varying degrees, and the solid dispersion prepared by different ratios of mixed materials has high dissolution rate and stability, which can overcome the problem of decreased dissolution rate in a single material formulation, and can significantly improve the oral bioavailability of vilazodone on an empty stomach.

According to the embodiments of the present invention, the vilazodone solid dispersion comprises vilazodone and a mixed material, the mixed material comprises copovidone and hypromellose. In some embodiments, the mixed material consists of copovidone and hypromellose.

According to the embodiments of the present invention, the mixed material comprises copovidone and hydroxypropyl methylcellulose, and the weight ratio of the copovidone to the hydroxypropyl methylcellulose is 1:1 to 3:1. In some embodiments, the weight ratio of the copovidone to the hydroxypropyl methylcellulose is 1:1. In some embodiments, the weight ratio of the copovidone to the hydroxypropyl methylcellulose is 2:1. In some embodiments, the weight ratio of the copovidone to the hydroxypropyl methylcellulose is 3:1.

According to the embodiments of the present invention, the vilazodone solid dispersion comprises vilazodone and the said mixed material, and the mixed material comprises povidone and hypromellose. In some embodiments, the mixed material consists of povidone and hypromellose.

According to the embodiments of the present invention, the mixed material comprises povidone and hydroxypropyl methylcellulose, and the weight ratio of the povidone to the hydroxypropyl methylcellulose is 1:2 to 2:1. In some embodiments, the weight ratio of the povidone to the hydroxypropyl methylcellulose is 1:2. In some embodiments, the weight ratio of the povidone to the hydroxypropyl methylcellulose is 1:1. In some embodiments, the weight ratio of the povidone to the hydroxypropyl methylcellulose is 2:1.

According to the embodiments of the present invention, the weight ratio of the vilazodone to the water-soluble polymer carrier material is 1:4 to 1:7. In some embodiments, the weight ratio of the vilazodone to the water-soluble polymer carrier material is 1:5. In some embodiments, the weight ratio of vilazodone to the water-soluble polymer carrier material is 1:6.

In some embodiments, the vilazodone solid dispersion comprises vilazodone, copovidone, and a surfactant.

In some embodiments, the vilazodone solid dispersion comprises vilazodone, povidone, and a surfactant.

In some embodiments, the vilazodone solid dispersion comprises vilazodone, hydroxypropyl methylcellulose, and a surfactant.

The second aspect of the present invention provides a method for preparing the vilazodone solid dispersion. The vilazolone solid dispersion can be prepared by a variety of techniques, including but not limited to spray-drying, hot melt extrusion, freeze-drying method, solvent evaporation method, co-precipitation method, supercritical fluid method, etc.

In some embodiments, the present invention adopts a hot melt extrusion method to prepare the vilazodone solid dispersion, which is simple to operate and easy to mass produce. In the prepared solid dispersion, vilazodone exists in an amorphous form, and the in vitro dissolution rate of the solid dispersion is significantly improved compared to the commercially formulation VIIBRYD®.

In other embodiments, the spray-drying method is used to prepare the solid dispersion of vilazodone in the present invention. The spray drying method includes the following steps: vilazodone and the carrier material weighed according to proportion are added to solvent and stirred to be dissolved, then spray-dried in a spray dryer, and the powder is collected to obtain the vilazodone solid dispersion. The solvent is an aqueous acetone solution, and the volume concentration of acetone is greater than 60% and less than or equal to 70%. When preparing a solid dispersion by spray-drying, it is important to find a safer solvent that can dissolve both the drug and the material. Only when the drug and the material are both dissolved, the drug and the material can fully interact to obtain a more stable solid dispersion. Through a large number of screenings and optimizing, a 60%˜70% acetone aqueous solution has been obtained as the solvent. Using the solvent, a high concentration and stable vilazodone solution can be prepared for spray-drying, and under this concentration, the solubilities of various carrier materials are also high, which can greatly increase the solid content of the solution, significantly improve the spray-drying efficiency, and reduce energy consumption. Of course, without considering energy consumption, a low concentration vilazodone solution prepared by a lower concentration acetone solution, or methanol, ethanol solution, etc., can also be used for spray-drying. In addition, when preparing the vilazodone spray-dried solution, in order to dissolve the vilazodone API and the carrier material better and faster, the dissolution can be accelerated by heating or ultrasound. In the present invention, vilazodone exists in an amorphous form when the solid dispersion is prepared by using 60% to 70% acetone aqueous solution as a solvent, and the in vitro dissolution rate is significantly higher than the commercially formulation (VIIBRYD®).

According to the embodiments of the present invention, the vilazodone solid dispersion can be prepared into formulation, wherein the dosage form of the formulation includes granule, powder, dry suspension, tablet or capsule. According to clinical treatment needs, the corresponding dosage forms mentioned above can be selectively prepared, which is convenient for patient to take and improves patient compliance. In order to prevent the moisture absorption and aging of the vilazodone solid dispersion, it can also be prepared into a coated tablet; in addition, in order to make vilazodone absorbed in the small intestine, it can also be prepared into an enteric coated tablet.

The third aspect of the present invention provides a use of the vilazodone solid dispersion, a use of the said vilazodone solid dispersion in the preparation of a vilazodone formulation. The vilazodone formulation comprises the vilazodone solid dispersion and pharmaceutically acceptable excipients. The pharmaceutically acceptable excipients include but are not limited to the followings: filler, such as lactose, sucrose, fructose, fructo-oligosaccharide, glucose, maltose, powdered sugar and other sugars, D-mannitol, erythritol, xylitol and other sugar alcohols, corn starch, potato starch, rice starch, some a starch and other starches, celluloses such as microcrystalline cellulose, calcium sulfate, calcium hydrogen phosphate, calcium carbonate and other inorganic salts; disintegrants, such as starch, microcrystalline cellulose, calcium carboxymethyl cellulose, croscarmellose sodium, polyvinylpolypyrrolidone, sodium carboxymethyl starch, low-substituted hydroxypropyl cellulose; lubricants, such as magnesium stearate, calcium stearate, sodium stearate fumarate, stearic acid, talc, polyethylene glycol, sucrose fatty acid ester, colloidal silicon dioxide.

The dosage form of vilazodone provided by the present invention includes tablet, capsule, granule, powder or dry suspension. According to clinical treatment needs, the corresponding dosage forms mentioned above can be selectively prepared, which is convenient for patient to take and improves patient compliance. In order to prevent the moisture absorption and aging of the vilazodone solid dispersion, it can also be prepared into a coated tablet; in addition, in order to make vilazodone absorbed in the small intestine, it can also be prepared into an enteric coated tablet.

In some embodiments, the present invention uses copovidone as carrier material and poloxamer 188 as surfactant to prepare the vilazodone solid dispersion, and after adding an appropriate amount of pharmaceutically acceptable excipients to prepare a tablet, under fasting condition, its oral bioavailability in Beagle dogs is significantly higher than that of the commercially formulation (VIIBRYD®), and under fasting and feeding conditions, the vilazodone solid dispersion herein is equivalent and eliminates the food effect, suggesting that the vilazodone solid dispersion herein can be taken under fasting and feeding conditions, which can significantly improve the convenience and compliance of patients in taking medication.

In some embodiments, the present invention further investigates the stability of vilazodone solid dispersion. Under accelerated condition (40° C., 75% RH), after 2 months of storage, vilazodone is still amorphous existing in the solid dispersion, and the in vitro dissolution rate has no significant change, indicating that the vilazodone solid dispersion prepared by the present invention has good stability and meets the requirements of industrial production.

The vilazodone solid dispersion provided herein can significantly improve the solubility and in vitro dissolution rate of vilazodone, thereby improving its fasting bioavailability; furthermore, the vilazodone solid dispersion provided herein also can eliminate the influence of food, and its oral bioavailability is equivalent under fasting and feeding conditions, suggesting that the formulation of vilazodone solid dispersion provided herein can be taken under fasting or feeding condition, which significantly improves the convenience and compliance of taking medicine for patients, and can be used for the treatment of moderate to severe depression.

### Definition of Terms

The invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described herein. In the event that one or more of the incorporated literature, patents, and similar materials differs from or contradicts this application, including but not limited to defined terms, term usage, described techniques, or the like, this application controls.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Unless defined otherwise, all technical and scientific terms used herein have the same meaning as is commonly understood by one skilled in the art to which this invention belongs. All patents and publications referred to herein are incorporated by reference in their entirety.

The term “comprise”, “include” or “contain” is an open expression, it means comprising the contents disclosed herein, but don't exclude other contents.

In the present invention, regardless of whether the words “about” or “approximately” are used, all numbers disclosed herein are approximate values. The value of each number may vary by less than 10%, or a reasonable difference that one skilled in the art would consider, such as 1%, 2%, 3%, 4% or 5%.

LC-MS-MS refers to liquid chromatography-mass spectrometry, and XRD refers to X-ray diffraction.

PVP refers to povidone, VA64 refers to copovidone, HPMC refers to hydroxypropyl methylcellulose, Labrasol refers to caprylic acid capric acid polyethylene glycol glyceride, Cremophor RH40 refers to polyoxyethylene 40 hydrogenated castor oil, T-80 refers to Tween-80, F68 refers to poloxamer 188, S-20 refers to Span-20, TPGS refers to (D) α-tocopherol polyethylene glycol 1000 succinate.

PEG refers to polyethylene glycol, Soluplus refers to polyethylene glycol/vinyl caprolactam/vinyl acetate copolymer, HPMCAS refers to hypromellose acetate succinate, and HPC refers to hydroxypropyl cellulose.

AUC refers to the area under the plasma concentration-time curve, and Cₘₐₓ refers to the peak plasma concentration.

M means mole, h means hour, g means gram, mm means millimeter, μm means micrometer, nm means nanometer, μl means microliter, min means minute, ° C. means Celsius degree, mg means milligram, mL means milliliter, rpm means revolutions per minute.

### BRIEF DESCRIPTION OF THE DRAWINGS

FIG. 1 shows the XRD patterns of vilazodone hydrochloride solid dispersions prepared with different proportion of VA64 of Example 3 (1).

FIG. 2 shows the XRD patterns of vilazodone hydrochloride solid dispersions prepared with different proportion of PVP of Example 3 (2).

FIG. 3 shows the XRD patterns of vilazodone hydrochloride solid dispersions prepared with different proportion of HPMC of Example 3 (3).

FIG. 4 shows the XRD patterns of vilazodone hydrochloride solid dispersions prepared with different mixed carriers of Example 3 (4).

FIG. 5 shows AUC of the formulation of vilazodone solid dispersion in Beagle dog of Example 7.

FIG. 6 shows the XRD pattern of vilazodone solid dispersion after 2 months of accelerated stability of Example 8.

### EXAMPLES

For better understanding the technical solutions herein, the present invention will be further described below in conjunction with specific embodiments. Unless otherwise specified, if specific technologies or conditions are not explicitly described in the following examples, those skilled in the art can proceed in accordance with common technologies or conditions in the field or the product instructions. If the medicines, reagents or instruments used are not specified by the manufacturers, they are all conventional products that are commercially available.

Wherein the reference formulation VIIBRYD® was vilazodone hydrochloride tablet with the strength of 10 mg and 40 mg developed by the forest laboratory.

Hydroxypropyl methylcellulose succinate (HPMCAS-LF and HPMCAS-MF) and polyethylene glycol 4000 (PEG-4000) were purchased from Ashland, USA; polyethylene glycol/vinyl caprolactam/vinyl acetate copolymer (Soluplus) and povidone (PVP) were purchased from BASF in Germany; copovidone (VA64) was purchased from BASF in Germany; hydroxypropyl methylcellulose (HPMC) was purchased from Ashland; hydroxypropyl cellulose (HPC) was purchased from Soda Corporation of Japan.

Tween-80 (T-80), Poloxamer 188 (F68), Span-20 (S-20), Polyoxyethylene 40 hydrogenated castor oil (Cremophor RH40), caprylic acid capric acid polyethylene glycol glyceride (Labrasol) and (D) α-tocopherol polyethylene glycol 1000 succinate (TPGS) were all commercially available surfactants.

The method for determining the content of vilazodone was high performance liquid chromatography (HPLC), and the specific conditions were: chromatographic conditions: detection wavelength was 242 nm, chromatographic column was kromasil 100-5 C18 (4.6 mm×150 mm, 5 μm), mobile phase was dipotassium phosphate and acetonitrile of 54:46 (V/V) with pH 6.0 and 0.02M, the flow rate was 1.0 ml/min, the injection volume was 10 μl, and the running time was 4.5 min.

### Example 1 Screening of Solvent

(1) Investigation of Single Solvent

10 mg of vilazodone hydrochloride solid powder was put into a 4 ml EP tube. 1 ml of dichloromethane, methanol, ethanol, acetone and ethyl acetate was added to each tube separately, and the tubes were sonicated in a water bath for 30 minutes. The dissolution of the powder was observed; after the samples were placed at room temperature for 24 h, the appearance of the samples was observed and whether there was precipitation was noted. The results were shown in Table 1.

TABLE 1
Dissolution of vilazodone in different organic solvents

| Solvent | Concentration | Phenomenon |
|---|---|---|
| Dichloromethane | 10 mg/ml | Insoluble, a lot of precipitation |
| Ethanol | 10 mg/ml | Insoluble, a little of precipitation |
| Methanol | 10 mg/ml | Insoluble, a little of precipitation |
| Acetone | 10 mg/ml | Insoluble, a little of precipitation |
| Ethyl acetate | 10 mg/ml | Insoluble, a lot of precipitation |

The above results showed that no single organic solvent can be used to prepare a 10 mg/ml vilazodone solution. In comparison, vilazodone hydrochloride has relatively good solubility in ethanol, methanol or acetone, and can be used as a solvent for subsequent optimization.

(2) Investigation of Ethanol Aqueous Solution

10 mg of vilazodone hydrochloride solid powder was put into a 4 ml EP tube. 1 ml of ethanol aqueous solution of different concentrations was added to each tube separately, and the tubes were sonicated in a water bath for 30 minutes. The dissolution of the powder was observed; after the samples were placed at room temperature for 24 h, the appearance of the samples was observed and whether there was precipitation was noted. The results were shown in Table 2.

TABLE 2
Dissolution of vilazodone hydrochloride
in ethanol aqueous solution

| Ethanol | Water | Vilazodone | |
|---|---|---|---|
| (ml) | (ml) | hydrochloride | Phenomenon |
| 0.1 | 0.9 | 10 mg | Insoluble, a lot of precipitation |
| 0.2 | 0.8 | 10 mg | Insoluble, a lot of precipitation |
| 0.3 | 0.7 | 10 mg | Insoluble, a lot of precipitation |
| 0.4 | 0.6 | 10 mg | Dissolved, precipitate after 1 day |
| 0.5 | 0.5 | 10 mg | Dissolved, place to precipitate |
| | | | slightly |
| 0.6 | 0.4 | 10 mg | Dissolved, precipitate after 1 day |
| 0.7 | 0.3 | 10 mg | Dissolved, place to precipitate |
| | | | slightly |
| 0.8 | 0.2 | 10 mg | Insoluble, a lot of precipitation |
| 0.9 | 0.1 | 10 mg | Insoluble, a lot of precipitation |

The above results showed that when using ethanol aqueous solutions of different concentrations to prepare a 10 mg/ml vilazodone hydrochloride solution, a highly concentrated stable solution cannot be obtained.

(3) Investigation of Methanol Aqueous Solution

30 mg of vilazodone hydrochloride solid powder was put into a 10 ml EP tube 3 ml of methanol aqueous solution of different concentrations was added to each tube separately, and the tubes were sonicated in a water bath for 30 minutes. The dissolution of the powders was observed; if the powder was dissolved quickly, a small amount of powder was continued to add to increase the concentration of the sample and was dissolved ultrasonically; finally, the solution samples were placed at room temperature for 24 hours to observe whether there was precipitation. The results were shown in Table 3.

TABLE 3
Dissolution of vilazodone hydrochloride
in methanol aqueous solution

| Methanol | Water | Vilazodone | |
|---|---|---|---|
| (ml) | (ml) | hydrochloride | Result |
| 0.3 | 2.7 | 30 mg | Insoluble, a lot of precipitation |
| 0.6 | 2.4 | 30 mg | Insoluble, a lot of precipitation |
| 0.9 | 2.1 | 30 mg | Insoluble, a lot of precipitation |
| 1.2 | 1.8 | 30 mg | Insoluble, a lot of precipitation |
| 1.5 | 1.5 | 30 mg | Dissolved, continue to add a small |
| | | | amount of powder to 15 mg/ml, |
| | | | insoluble |
| 1.8 | 1.2 | 30 mg | Continue to add a small amount of |
| | | | powder to 15 mg/ml, dissolved, |
| | | | place to precipitate |
| 2.1 | 0.9 | 30 mg | Continue to add a small amount of |
| | | | powder to 15 mg/ml, insoluble |
| 2.4 | 0.6 | 30 mg | Dissolved, continue to add a small |
| | | | amount of powder to 15 mg/ml, |
| | | | insoluble |
| 2.7 | 0.3 | 30 mg | Dissolved, continue to add a small |
| | | | amount of powder to 15 mg/ml, |
| | | | insoluble |

The above results showed that a 10 mg/ml vilazodone hydrochloride solution could be successfully prepared by using a methanol aqueous solution with a concentration of more than 50%, but when the drug concentration continued to increase, precipitation occurred and could not be dissolved, and high concentration and stable vilazodone solution could not be obtained to meet the experimental requirements.

(4) Investigation of Acetone Aqueous Solution

10 mg of vilazodone hydrochloride solid powder was put into a 4 ml EP tube. 1 ml of acetone aqueous solution of different concentrations was added to each tube separately, and the tubes were sonicated in a water bath for 30 minutes. The dissolution of the powder was observed; if the powder was dissolved quickly, a small amount of powder was continued to add to increase the concentration of the sample and was dissolved ultrasonically; finally, the solution samples were placed at room temperature for 24 hours to observe whether there was precipitation. The results were shown in Table 4.

TABLE 4
Dissolution of vilazodone hydrochloride
in acetone aqueous solution

| Acetone | Water | Vilazodone | |
|---|---|---|---|
| (ml) | (ml) | hydrochloride | Phenomenon |
| 0.1 | 0.9 | 10 mg | Insoluble, a lot of precipitation |
| 0.2 | 0.8 | 10 mg | Insoluble, a lot of precipitation |
| 0.3 | 0.7 | 10 mg | Insoluble, a lot of precipitation |
| 0.4 | 0.6 | 10 mg | Dissolved completely and place to |
| | | | precipitate |
| 0.5 | 0.5 | 10 mg | Dissolved completely and place to |
| | | | precipitate |
| 0.6 | 0.4 | 10 mg | Dissolved completely, increase the |
| | | | concentration to 40 mg/ml, without |
| | | | precipitation |
| 0.7 | 0.3 | 10 mg | Dissolved completely, increase the |
| | | | concentration to 40 mg/ml, without |
| | | | precipitation |
| 0.8 | 0.2 | 10 mg | Dissolved completely and place to |
| | | | precipitate |
| 0.9 | 0.1 | 10 mg | Insoluble, a lot of precipitation |

The above results showed that the use of about 60% to 70% acetone aqueous solution can successfully prepare a stable high-concentration vilazodone hydrochloride solution, which could meet the requirements of subsequent experiments.

(5) Investigation of the Solubility of Carrier Materials in Acetone Solution

5 ml of acetone solution (50%, 60%, 70%, 80% volume concentration) was put into a 5 mL BD tube respectively, and about 250 mg of carrier material was added. The mixture was stirred to be dissolved in a magnetic stirrer. If the solution became clear, the carrier material was continued to add and stirred to be dissolved until the solution became turbid. The amount of the material added was recorded. The results were shown in Table 5.

TABLE 5
The dissolution of different carrier materials in acetone aqueous solution

| | 50% acetone solution | 60% acetone solution | 70% acetone solution | 80% acetone solution |
|---|---|---|---|---|
| PVP | Clear, solubility >160 mg/ml | Clear, solubility >170 mg/ml | Turbid, dissolved by heating | Turbid, insoluble by heating |
| HPMC | Clear, solubility >110 mg/ml | Clear, solubility >120 mg/ml | Clear, solubility >120 mg/ml | Clear, sticky >120 mg/ml |
| HPC | Clear, solubility >130 mg/ml | Clear, solubility >130 mg/ml | Clear, solubility >130 mg/ml | Clear, solubility >130 mg/ml |
| HPMCAS-LF | Turbid, insoluble by heating | Clear, solubility >100 mg/ml | Clear, solubility >100 mg/ml | Clear, solubility >100 mg/ml |
| VA64 | Clear, solubility >150 mg/ml | Clear, solubility >150 mg/ml | Clear, solubility >150 mg/ml | Clear, solubility >150 mg/ml |

The above results showed that in order to simultaneously satisfy the dissolution of the drug and the carrier material and obtain a suitable solution viscosity for spray-drying, the concentration of the acetone solution was 60% to 70%. Of course, if lower concentration of drugs and materials were used, the concentration range of acetone could be further expanded to 50% to 80%, or greater.

### Example 2 Screening of Different Carrier Materials

(1) Determination of Dissolution Rate of Commercially Formulation VIIBRYD®

6 VIIBRYD® tablets (10 mg/40 mg) were taken, and the dissolution rate in the medium of 0.1M HCl, pH3.1 and pH6.8 was determined respectively according to the United States Pharmacopeia II (USP II), with 900 mL of medium, 60 rpm speed as the conditions. After the start of the test, 10 ml of sample at 5 min, 10 min, 15 min, 20 min, 30 min and 45 min time points was taken respectively, and 10 ml of fresh dissolution medium was immediately supplemented with. The test was continued. The sample was passed through a 0.45 μm filter membrane, and an appropriate amount of the filtrate was taken. The drug content in the sample was determined by HPLC method, and the cumulative dissolution rate was calculated at each time point. The results were shown in Table 6.

TABLE 6
Test results of in vitro dissolution rate of VIIBRYD ® tablets

| | | | | | | |
|---|---|---|---|---|---|---|
| Dissolution | | | | | | |
| media | 5 min | 10 min | 15 min | 20 min | 30 min | 45 min |

Dissolution rate of 10 mg VIIBRYD ® tablets in different dissolution media

| | | | | | | |
|---|---|---|---|---|---|---|
| PH 3.1 | N/A | 87 ± 2.3 | 93 ± 1 | 93 ± 1 | 93 ± 1.7 | 93 ± 0.6 |
| PH 6.8 | N/A | 2 ± 0.6 | 4 ± 0.6 | 4 ± 0.6 | 5 ± 0 | 6 ± 0.6 |
| 0.1 MHCl | N/A | 25 ± 2.9 | 33 ± 2.1 | 38 ± 1.2 | 46 ± 4.2 | 56 ± 1.5 |

Dissolution rate of 40 mg VIIBRYD ® tablets in different dissolution media

| | | | | | | |
|---|---|---|---|---|---|---|
| PH 3.1 | 78 ± 6.03 | 95 ± 1.73 | 98 ± 1.15 | 99 ± 0.58 | 100 ± 0.58 | 100 ± 0.58 |
| PH 6.8 | 3 ± 0.58 | 4 ± 0.00 | 4 ± 0.00 | 4 ± 0.00 | 4 ± 0.00 | 4 ± 0.58 |
| 0.1 MHCl | 14 ± 3.00 | 22 ± 1.15 | 24 ± 4.04 | 29 ± 2.52 | 26 ± 10.15 | 15 ± 2.65 |

(2) Investigation of the Types of Carrier Materials

According to the formulations in Table 7, each carrier material and vilazodone hydrochloride powder were put into a beaker, and 60% acetone aqueous solution was added. The mixture was stirred to be dissolved under the heating condition of a water bath at 40° C., and spray-dried in a Buchi spray drying apparatus (inlet air temperature: 170° C., outlet air temperature: 95° C., rotation speed: 15 rpm) to obtain a spray-dried powder The above-mentioned spray-dried powder and microcrystalline cellulose was mixed at a ratio of 1:3, and the dissolution rate of 40 mg of samples in pH 6.8 medium was tested according to the method under “Determination of dissolution rate of commercial available formulation”. The results were shown in Table 8.

TABLE 7
Different carrier material formulations

| Components | Formulation -1 | | Formulation -2 | | Formulation -3 | | Formulation -4 | | Formulation -5 | | Formulation -6 | | Formulation -7 | |
|---|---|---|---|---|---|---|---|---|---|---|---|---|---|---|
| Vilazodone hydrochloride | 10 | g | 10 | g | 10 | g | 10 | g | 10 | g | 10 | g | 10 | g |
| Material types | HPMCAS-LF | | HPMCAS-MF | | Soluplus | | PVP | | HPMC | | HPC | | VA64 | |
| Material weight | 60 | g | 60 | g | 60 | g | 60 | g | 60 | g | 60 | g | 60 | g |
| 60% acetone solution | 1000 | ml | 1000 | ml | 1000 | ml | 1000 | ml | 1000 | ml | 1000 | ml | 1000 | ml |

TABLE 8
Results of dissolution rate of solid dispersions prepared
with different carrier materials in pH 6.8 media

| Time | 5 min | 10 min | 15 min | 20 min | 30 min | 45 min |
|---|---|---|---|---|---|---|
| VIIBRYD ® | 3 ± 0.58 | 4 ± 0.00 | 4 ± 0.00 | 4 ± 0.00 | 4 ± 0.00 | 4 ± 0.58 |
| Formulation -1 | 34 ± 1.54 | 37 ± 2.52 | 39 ± 2.52 | 37 ± 1.00 | 37 ± 1.53 | 36 ± 2.08 |
| Formulation -2 | 26 ± 2.31 | 30 ± 1.00 | 31 ± 1.53 | 32 ± 0.00 | 32 ± 1.00 | 34 ± 0.58 |
| Formulation -3 | 3 ± 0.58 | 6 ± 1.00 | 8 ± 1.53 | 11 ± 1.53 | 15 ± 1.53 | 21 ± 2.31 |
| Formulation -4 | 67 ± 2.65 | 70 ± 1.53 | 74 ± 3.46 | 74 ± 2.08 | 66 ± 9.61 | 61 ± 4.16 |
| Formulation -5 | 72 ± 2.05 | 77 ± 2.08 | 78 ± 2.08 | 78 ± 0.00 | 77 ± 3.00 | 73 ± 2.65 |
| Formulation -6 | 32 ± 1.53 | 39 ± 10.58 | 50 ± 12.06 | 45 ± 14.84 | 46 ± 25.79 | 29 ± 11.55 |
| Formulation -7 | 48 ± 4.04 | 51 ± 3.61 | 53 ± 3.61 | 54 ± 3.21 | 54 ± 3.61 | 55 ± 3.51 |

The above results showed that the dissolution rates of the vilazodone hydrochloride solid dispersion prepared by using copovidone (VA64), povidone (PVP) or hydroxypropyl methylcellulose (HPMC) were relatively high in the simulated intestinal juice (pH 6.8 medium). Under high strength (40 mg) condition, the dissolution platform was greater than 500%, and it was far greater than the dissolution rate of the reference formulation VIIBRYD®, which could significantly increase the oral bioavailability of vilazodone under fasting condition and used as the basis for subsequent formulation optimization. However, under the condition of high dissolution rate (70%), the dissolution rate of some formulations showed a trend of decline.

(3) Investigation of Mixed Carrier Materials

According to the formulations in Table 9, each carrier material and vilazodone hydrochloride powder were put into a beaker, and 60% acetone aqueous solution was added. The mixture was stirred to be dissolved under the heating condition of a water bath at 50° C., and spray-dried in a Buchi spray drying apparatus to obtain a spray-dried powder; wherein the formulation 11 included low melting point material PEG4000, the inlet air temperature was 95° C., the outlet air temperature was 50° C., and the rotation speed was 20 rpm; the inlet air temperature of the other formulations was 160° C., the outlet air temperature was 85° C., and the rotation speed was 20 rpm. The above-mentioned spray-dried powder and microcrystalline cellulose were mixed at a ratio of 1:3, and the dissolution rate of 40 mg of samples in pH 6.8 medium was tested according to the method under “Determination of dissolution rate of commercial available formulation”. The results were shown in Table 10.

TABLE 9
Mixture of carrier material formulation

| Component | Formulation -8 | | Formulation -9 | | Formulation -10 | | Formulation -11 | |
|---|---|---|---|---|---|---|---|---|
| Vilazodone hydrochloride | 10 | g | 10 | g | 10 | g | 10 | g |
| VA64 | 20 | g | 30 | g | — | | — | |
| PVP | 40 | g | — | | 30 | g | 24 | g |
| HPMC | — | | 30 | g | 30 | g | — | |
| PEG-4000 | — | | — | | — | | 26 | g |
| 60% acetone aqueous solution | 1000 | ml | 1000 | ml | 1000 | ml | 1000 | ml |

TABLE 10
Results of dissolution rate of solid dispersions prepared
by mixed carrier materials in pH 6.8 media

| Time | 5 min | 10 min | 15 min | 20 min | 30 min | 45 min |
|---|---|---|---|---|---|---|
| VIIBRYD ® | 3 ± 0.58 | 4 ± 0.00 | 4 ± 0.00 | 4 ± 0.00 | 4 ± 0.00 | 4 ± 0.58 |
| Formulation -8 | 48 ± 1.53 | 54 ± 0.58 | 55 ± 1.15 | 57 ± 1.00 | 58 ± 0.71 | 63 ± 0.00 |
| Formulation -9 | 62 ± 1.15 | 70 ± 2.08 | 75 ± 1.00 | 75 ± 2.52 | 77 ± 2.08 | 75 ± 1.00 |
| Formulation -10 | 71 ± 4.04 | 77 ± 1.00 | 78 ± 1.53 | 76 ± 1.73 | 76 ± 2.52 | 75 ± 1.53 |
| Formulation -11 | 53 ± 1.41 | 56 ± 7.07 | 61 ± 2.12 | 64 ± 0.70 | 72 ± 9.19 | 58 ± 14.14 |

The above results showed that the dissolution rate of vilazodone hydrochloride solid dispersion prepared by mixed materials in simulated intestinal juice (pH 6.8 medium) was relatively high, and was far greater than the dissolution rate of the reference formulation VIIBRYD®; when PVP+PEG4000 were used as the material, the dissolution rate tended to decrease. When VA64+HPMC or PVP+HPMC were used as the material, the prepared vilazodone hydrochloride solid dispersion (40 mg strength) had the highest dissolution, which were both greater than 7000, and relatively stable (variation range was less than 50%), and no crystallization or decrease in dissolution rate occurred, which can significantly increase the oral bioavailability of vilazodone under fasting conditions.

### Example 3 Investigation of the Carrier Material Proportion

(1) Investigation of the Proportion of Copovidone

According to the formulations in Table 11, different proportions of copovidone (VA64) and vilazodone hydrochloride powder were put into a beaker, and 70% acetone aqueous solution was added. The mixture was stirred to be dissolved and spray dried in a Buchi spray dryer (inlet air temperature: 170° C., outlet air temperature: 90° C., rotation speed: 20 rpm) to obtain a spray-dried powder. The spray-dried powder and microcrystalline cellulose were mixed in a ratio of 1:3. According to the dissolution rate test method described in Example 2 (1), the dissolution rate of 40 mg sample in pH 6.8 medium was detected. The results were shown in the table 12.

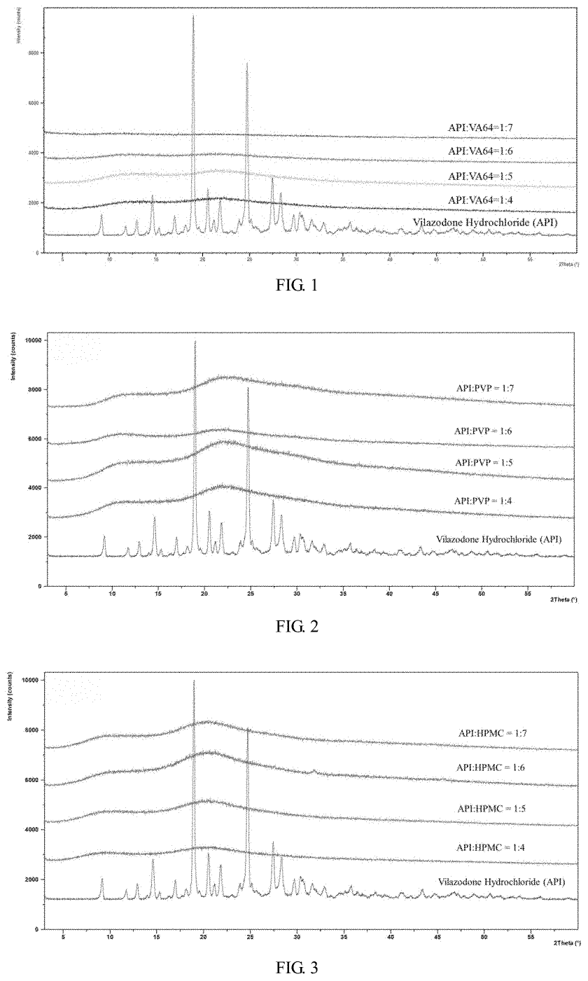

In addition, an appropriate amount of the spray-dried powder of the above formulation was taken, and the X-ray diffraction peak of vilazodone was measured in an X-ray diffractometer (XRD). The specific spectrum was shown in FIG. 1.

TABLE 11
Different copovidone proportion formulations

| Components | Formulation -12 | | Formulation -13 | | Formulation -14 | | Formulation -15 | |
|---|---|---|---|---|---|---|---|---|
| Vilazodone hydrochloride | 10 | g | 10 | g | 10 | g | 10 | g |
| Copovidone | 40 | g | 50 | g | 60 | g | 70 | g |
| 70% acetone aqueous solution | 1000 | ml | 1000 | ml | 1000 | ml | 1000 | ml |

TABLE 12
Results of in vitro dissolution rate of solid dispersions prepared
with different proportions of copovidone in pH 6.8 media

| Time | 5 min | 10 min | 15 min | 20 min | 30 min | 45 min |
|---|---|---|---|---|---|---|
| VIIBRYD ® | 3 ± 0.58 | 4 ± 0.00 | 4 ± 0.00 | 4 ± 0.00 | 4 ± 0.00 | 4 ± 0.58 |
| Formulation -12 | 32 ± 9.54 | 25 ± 12.53 | 39 ± 5.00 | 43 ± 9.45 | 44 ± 8.19 | 51 ± 3.79 |
| Formulation -13 | 31 ± 13.11 | 40 ± 8.89 | 43 ± 7.94 | 44 ± 6.08 | 45 ± 3.61 | 45 ± 2.52 |
| Formulation -14 | 48 ± 4.04 | 51 ± 3.61 | 53 ± 3.61 | 54 ± 3.21 | 54 ± 3.61 | 55 ± 3.51 |
| Formulation -15 | 29 ± 7.09 | 40 ± 4.51 | 44 ± 4.04 | 46 ± 3.46 | 49 ± 3.61 | 51 ± 3.61 |

The above results showed that when the weight ratio of vilazodone hydrochloride to copovidone (VA64) was less than or equal to 1:4, the drug existed in the solid dispersion in an amorphous form, and the dissolution rate in the simulated intestinal juice (pH 6.8 medium) was significantly higher than the reference formulation VIIBRYD® (40 mg strength), and stable, which could significantly increase the oral bioavailability of vilazodone under fasting condition. However, as the amount of material increased, the dissolution rate of the drug did not increase significantly.

(2) Investigation of the Proportion of Povidone

According to the formulations in Table 13, different proportion of povidone (PVP) and vilazodone hydrochloride powder were put into a beaker, and 60% acetone aqueous solution was added. The mixture was stirred to be dissolved and spray-dried in a Buchi spray dryer (inlet air temperature: 170° C., outlet air temperature: 90° C., rotation speed: 20 rpm) to obtain a spray-dried powder. The spray-dried powder and microcrystalline cellulose were mixed in a ratio of 1:3. According to the dissolution rate test method described in Example 2 (1), the dissolution rate of 40 mg sample in pH 6.8 medium was detected. The results were shown in the table 14.

In addition, an appropriate amount of the spray-dried powder of the above formulations was taken in an X-ray diffractometer (XRD) to detect the X-ray diffraction peak of vilazodone. The specific spectrum was shown in FIG. 2.

TABLE 13
Different povidone proportion formulations

| Components | Formulation -16 | | Formulation -17 | | Formulation -18 | | Formulation -19 | |
|---|---|---|---|---|---|---|---|---|
| Vilazodone hydrochloride | 10 | g | 10 | g | 10 | g | 10 | g |
| Povidone | 40 | g | 50 | g | 60 | g | 70 | g |
| 60% acetone aqueous solution | 1000 | ml | 1000 | ml | 1000 | ml | 1000 | ml |

TABLE 14
Results of in vitro dissolution rate of solid dispersions prepared
with different proportion of povidone materials in pH 6.8 media.

| Time | 5 min | 10 min | 15 min | 20 min | 30 min | 45 min |
|---|---|---|---|---|---|---|
| VIIBRYD ® | 3 ± 0.58 | 4 ± 0.00 | 4 ± 0.00 | 4 ± 0.00 | 4 ± 0.00 | 4 ± 0.58 |
| Formulation -16 | 65 ± 6.35 | 64 ± 2.89 | 71 ± 2.00 | 68 ± 1.53 | 66 ± 1.15 | 56 ± 1.00 |
| Formulation -17 | 69 ± 2.65 | 65 ± 10.58 | 54 ± 15.53 | 52 ± 14.19 | 52 ± 3.00 | 55 ± 1.53 |
| Formulation -18 | 67 ± 2.65 | 70 ± 1.53 | 74 ± 3.46 | 74 ± 2.08 | 66 ± 9.61 | 61 ± 4.16 |
| Formulation -19 | 69 ± 1.73 | 60 ± 8.74 | 32 ± 24.43 | 40 ± 14.64 | 30 ± 25.51 | 35 ± 1.73 |

The above results showed that when the weight ratio of vilazodone hydrochloride to povidone (PVP) was less than or equal to 1:4, the drug existed in the solid dispersion in an amorphous form, and the dissolution rate in the simulated intestinal juice (pH 6.8 medium) was significantly higher than the reference formulation VIIBRYD® (40 mg strength), suggesting that it can significantly increase the oral bioavailability of vilazodone under fasting condition. However, the solid dispersions prepared with different ratios of povidone all showed a trend of decline in dissolution, showing that they were unstable.

(3) Investigation of the Proportion of Hydroxypropyl Methylcellulose

According to the formulations in Table 15, different proportion of hydroxypropyl methylcellulose (HPMC) and vilazodone hydrochloride powder were put into a beaker, and 60% acetone aqueous solution was added. The mixture was stirred to be dissolved and spray-dried in a Buchi spray drying apparatus (inlet air temperature: 170° C., outlet air temperature: 95° C., rotation speed: 20 rpm) to obtain a spray-dried powder. The spray-dried powder and microcrystalline cellulose were mixed in a ratio of 1:3. According to the dissolution rate test method described in Example 2 (1), the dissolution rate of 40 mg sample in pH 6.8 medium was detected. The results were shown in the table 16.

In addition, an appropriate amount of the spray-dried powder of the above formulation was taken in an X-ray diffractometer (XRD), and the X-ray diffraction peak of vilazodone was detected. The specific spectrum was shown in FIG. 3.

TABLE 15
Different proportion formulations of hydroxypropyl methylcellulose

| Components | Formulation -20 | | Formulation -21 | | Formulation -22 | | Formulation -23 | |
|---|---|---|---|---|---|---|---|---|
| Vilazodone hydrochloride | 10 | g | 10 | g | 10 | g | 10 | g |
| HPMC | 40 | g | 50 | g | 60 | g | 70 | g |
| 60% acetone aqueous solution | 1200 | ml | 1200 | ml | 1200 | ml | 1200 | ml |

TABLE 16
Results of in vitro dissolution rate of solid dispersions
prepared with different proportion of HPMC in pH 6.8 media

| Time | 5 min | 10 min | 15 min | 20 min | 30 min | 45 min |
|---|---|---|---|---|---|---|
| VIIBRYD ® | 3 ± 0.58 | 4 ± 0.00 | 4 ± 0.00 | 4 ± 0.00 | 4 ± 0.00 | 4 ± 0.58 |
| Formulation -20 | 38 ± 10.79 | 68 ± 3.06 | 70 ± 3.79 | 73 ± 4.58 | 72 ± 1.53 | 65 ± 3.51 |
| Formulation -21 | 45 ± 15.18 | 71 ± 5.03 | 75 ± 2.52 | 73 ± 2.08 | 76 ± 1.73 | 62 ± 3.61 |
| Formulation -22 | 46 ± 10.82 | 66 ± 4.93 | 79 ± 2.52 | 78 ± 0.00 | 77 ± 3.51 | 64 ± 8.08 |
| Formulation -23 | 59 ± 2.00 | 70 ± 5.13 | 71 ± 5.69 | 80 ± 2.08 | 76 ± 1.53 | 67 ± 2.65 |

The above results showed that when the weight ratio of vilazodone hydrochloride to hydroxypropyl methylcellulose (HPMC) was less than or equal to 1:4, the drug existed in an amorphous form in the solid dispersion, and the dissolution rate in the simulated intestinal juice (pH 6.8 medium) was significantly higher than that of the reference formulation VIIBRYD® (40 mg strength), which could significantly increase the oral bioavailability of vilazodone under fasting condition. However, the solid dispersions prepared with different proportions of HPMC showed a decline in the later period of the dissolution rate determination.

(4) Investigation of the Proportion of Mixed Carrier Materials

According to the formulations in Table 17, different proportions of carrier materials and vilazodone hydrochloride powder were put into a beaker, and 60% acetone aqueous solution was added. The mixture was stirred to be dissolved under heating in a water bath at 50° C. and spray-dried in a Buchi spray drying apparatus (inlet air temperature: 150° C., outlet air temperature: 85° C., rotation speed: 20 rpm) to obtain a spray-dried powder. The above-mentioned spray-dried powder and microcrystalline cellulose were mixed at a ratio of 1:3, and the dissolution rate of 40 mg samples in pH 6.8 medium was tested according to the method under “Determination of dissolution rate of dommercial available formulation”. The results were shown in Table 18.

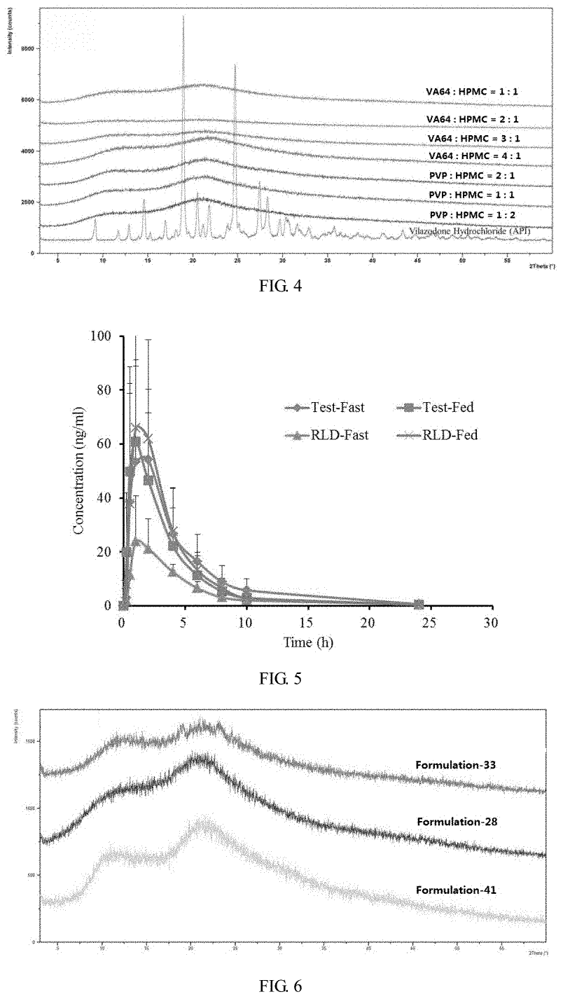

In addition, an appropriate quantity of the spray-dried powder of the above formulations were taken in an X-ray diffractometer (XRD), and the X-ray diffraction peaks of vilazodone hydrochloride were detected. The specific spectrum was shown in FIG. 4.

TABLE 17
Formulations of carrier materials mixed in different proportions

| Components | Formulation -24 | | Formulation -25 | | Formulation -26 | | Formulation -27 | | Formulation -28 | | Formulation -29 | |
|---|---|---|---|---|---|---|---|---|---|---|---|---|
| Vilazodone hydrochloride | 10 | g | 10 | g | 10 | g | 10 | g | 10 | g | 10 | g |
| Material types | PVP-K30 + HPMC-E5 | | | | | | VA64 + HPMC-E5 | | | | | |
| Material | 20 g + 40 g | | 30 g + 30 g | | 40 g + 20 g | | 37.5 g + 12.5 g | | 33 g + 17 g | | 25 g + 25 g | |
| 60% acetone solution | 1000 | ml | 1000 | ml | 1000 | ml | 1000 | ml | 1000 | ml | 1000 | ml |

TABLE 18
Results of dissolution rate of solid dispersions prepared by mixed
materials in different proportions in pH 6.8 media (Mean ± SD)

| Time | 5 min | 10 min | 15 min | 20 min | 30 min | 45 min |
|---|---|---|---|---|---|---|
| VIIBRYD ® | 3 ± 0.58 | 4 ± 0.00 | 4 ± 0.00 | 4 ± 0.00 | 4 ± 0.00 | 4 ± 0.58 |
| Formulation -24 | 63 ± 2.89 | 72 ± 4.00 | 73 ± 1.00 | 75 ± 2.08 | 71 ± 1.73 | 71 ± 1.73 |
| Formulation -25 | 71 ± 4.04 | 77 ± 1.00 | 78 ± 1.53 | 76 ± 1.73 | 76 ± 2.52 | 75 ± 1.53 |
| Formulation -26 | 56 ± 4.58 | 63 ± 5.13 | 69 ± 5.51 | 69 ± 3.46 | 70 ± 1.00 | 72 ± 2.08 |
| Formulation -27 | 65 ± 1.15 | 71 ± 4.51 | 70 ± 3.06 | 71 ± 3.51 | 74 ± 2.08 | 72 ± 3.79 |
| Formulation -28 | 68 ± 2.08 | 73 ± 5.20 | 76 ± 2.08 | 78 ± 5.51 | 75 ± 1.53 | 76 ± 1.53 |
| Formulation -29 | 67 ± 3.06 | 77 ± 0.58 | 75 ± 2.89 | 77 ± 2.08 | 76 ± 2.08 | 77 ± 2.08 |

The above results showed that when the vilazodone hydrochloride solid dispersion was prepared by different proportions of mixed materials, the drug existed in the solid dispersion in an amorphous form, and its dissolution rate in the simulated intestinal juice (pH 6.8 medium) was high and stable (dissolution rate greater than 70%, dissolution platform fluctuations within 5%), and it was significantly higher than the reference formulation VIIBRYD® (40 mg strength), which could significantly increase the oral bioavailability of vilazodone under fasting condition.

### Example 4 Investigation of Surfactant

(1) Investigation of Copovidone+Surfactant

According to the formulations in Table 19, each carrier material, surfactant and vilazodone hydrochloride powder were put into a beaker, and 60% acetone aqueous solution was added. The mixture was stirred to be dissolved and spray-dried in a small Buchi spray drying instrument (inlet air temperature: 170° C., outlet air temperature: 90° C., rotation speed: 20 rpm) to obtain a spray-dried powder. The spray-dried powder and microcrystalline cellulose were mixed in a ratio of 1:3. According to the dissolution test method described in Example 2 (1), the dissolution rate of 40 mg sample in pH 6.8 medium was detected. The results were shown in the table 20.

TABLE 19
Formulations of copovidone and different surfactants

| Components | Formulation -30 | Formulation -31 | | Formulation -32 | | Formulation -33 | | Formulation -34 | | Formulation -35 | |
|---|---|---|---|---|---|---|---|---|---|---|---|
| Vilazodone hydrochloride | 10 g | | | | | | | | | | |
| Copovidone | 60 g | | | | | | | | | | |
| Surfactant | — | T-80 | | Span-20 | | F68 | | Labrasol | | TPGS | |
| weight of surfactant | — | 7.78 | g | 7.78 | g | 7.78 | g | 7.78 | g | 7.78 | g |
| 60% acetone solution | 1000 ml | 1000 | ml | 1000 | ml | 1000 | ml | 1000 | ml | 1000 | ml |

TABLE 20
Results of dissolution rate of formulations of copovidone
and different surfactant in pH 6.8 media (Mean ± SD)

| Time | 5 min | 10 min | 15 min | 20 min | 30 min | 45 min |
|---|---|---|---|---|---|---|
| VIIBRYD ® | 3 ± 0.58 | 4 ± 0.00 | 4 ± 0.00 | 4 ± 0.00 | 4 ± 0.00 | 4 ± 0.58 |
| Formulation -30 | 48 ± 4.04 | 51 ± 3.61 | 53 ± 3.61 | 54 ± 3.21 | 54 ± 3.61 | 55 ± 3.51 |
| Formulation -31 | 62 ± 2.17 | 57 ± 2.89 | 34 ± 3.45 | 13 ± 4.12 | 8 ± 3.94 | 8 ± 5.02 |
| Formulation -32 | 67 ± 3.33 | 64 ± 5.21 | 62 ± 2.78 | 60 ± 1.49 | 57 ± 2.04 | 42 ± 1.58 |
| Formulation -33 | 65 ± 3.21 | 66 ± 4.00 | 66 ± 2.52 | 66 ± 3.21 | 69 ± 3.06 | 70 ± 2.08 |
| Formulation -34 | 76 ± 1.00 | 76 ± 1.53 | 68 ± 1.00 | 38 ± 2.89 | 9 ± 1.00 | 4 ± 0.00 |
| Formulation -35 | 66 ± 1.53 | 65 ± 7.09 | 67 ± 4.04 | 58 ± 7.09 | 56 ± 3.06 | 29 ± 4.51 |

The above results showed that on the basis of the copovidone material, the dissolution rate of the prepared solid dispersion of vilazodone was greatly improved after adding different surfactants, but most of the formulations appeared unstable and the dissolution rate declined. When poloxamer 188 (F68) was added, the dissolution rate was significantly improved and stable, and was significantly higher than that of the reference formulation VIIBRYD® (40 mg strength), which could significantly increase the oral bioavailability of vilazodone under fasting condition.

(2) Investigation of Povidone or Hydroxypropyl Methylcellulose and Surfactant

According to the formulations in Table 21, each carrier material, surfactant and vilazodone hydrochloride powder were put into a beaker, and 60% acetone aqueous solution was added. The mixture was stirred to be dissolved and spray-dried in a small Buchi spray drying instrument (inlet air temperature: 170° C., outlet air temperature: 95° C., rotation speed: 15 rpm) to obtain a spray-dried powder. The spray-dried powder and microcrystalline cellulose were mixed in a ratio of 1:3. According to the dissolution rate test method described in Example 2 (1), the dissolution rate of 40 mg sample in pH 6.8 medium was detected. The results were shown in the table 22.

TABLE 21
Different materials + surfactant formulation

| Components | Formulation -36 | Formulation -37 | | Formulation -38 | | Formulation -39 |
|---|---|---|---|---|---|---|
| Vilazodone hydrochloride | 10 g | 10 | g | 10 | g | 10 g |
| Carrier material | HPMC | | | | | |
| Material weight | 60 g | 60 | g | 60 | g | 60 g |
| Surfactant | — | Cremoph or RH40 | | Labrasol | | F68 |
| Surfactant weight | — | 3.68 | g | 3.68 | g | 3.68 |

| Components | Formulation -40 | Formulation -41 | Formulation -42 | Formulation -43 |
|---|---|---|---|---|
| Vilazodone hydrochloride | 10 g | 10 g | 10 g | 10 g |

| | |
|---|---|
| Carrier material | Povidone |

| | | | | |
|---|---|---|---|---|
| Material weight | 60 g | 60 g | 60 g | 60 g |
| Surfactant | — | Span-20 | F68 | TPGS |
| Surfactant weight | — | 3.68 | 7.78 | 3.68 |

TABLE 22
Results of dissolution rate of different materials and
surfactant formulations in pH 6.8 media (Mean ± SD)

| Time | 5 min | 10 min | 15 min | 20 min | 30 min | 45 min |
|---|---|---|---|---|---|---|
| VIIBRYD ® | 3 ± 0.58 | 4 ± 0.00 | 4 ± 0.00 | 4 ± 0.00 | 4 ± 0.00 | 4 ± 0.58 |
| Formulation -36 | 46 ± 10.82 | 66 ± 4.93 | 79 ± 2.52 | 78 ± 0.00 | 77 ± 3.51 | 64 ± 8.08 |
| Formulation -37 | 56 ± 2.08 | 77 ± 1.15 | 83 ± 1.00 | 82 ± 3.00 | 85 ± 2.08 | 83 ± 2.89 |
| Formulation -38 | 72 ± 4.04 | 81 ± 1.15 | 79 ± 4.16 | 81 ± 0.58 | 79 ± 0.58 | 78 ± 2.08 |
| Formulation -39 | 57 ± 18.03 | 73 ± 2.65 | 77 ± 1.53 | 75 ± 2.65 | 75 ± 4.04 | 31 ± 4.93 |
| Formulation -40 | 67 ± 2.65 | 70 ± 1.53 | 74 ± 3.46 | 74 ± 2.08 | 66 ± 9.61 | 61 ± 4.16 |
| Formulation -41 | 73 ± 4.16 | 77 ± 2.08 | 78 ± 2.08 | 78 ± 2.65 | 75 ± 1.15 | 75 ± 2.52 |
| Formulation -42 | 74 ± 4.93 | 75 ± 4.16 | 77 ± 2.31 | 77 ± 2.08 | 74 ± 3.00 | 74 ± 3.21 |
| Formulation -43 | 82 ± 2.00 | 84 ± 0.58 | 79 ± 1.53 | 79 ± 4.04 | 31 ± 24.54 | 22 ± 5.86 |

The above results showed that on the basis of the HPMC carrier material, when Cremophor RH40 or Labrasol surfactant was added, the dissolution rate of the solid dispersion was high and stable, but when the F68 surfactant was added, the dissolution rate was unstable and appeared decrease; on the basis of povidone material, when Span-20 or F68 surfactant was added, the dissolution rate of the solid dispersion was high and stable, while when TPGS surfactant was added, the dissolution rate was unstable and decreased. The above results suggested that adding a specific surfactant to the formulation can further increase the oral bioavailability of vilazodone under fasting condition.

### Example 5 Investigation of the Proportion of Surfactant

According to the formulations in Table 23, each carrier material, surfactant and vilazodone hydrochloride powder were put into a beaker, and 60% acetone aqueous solution was added. The mixture was stirred to be dissolved and spray-dried in a small Buchi spray drying instrument (inlet air temperature: 170° C., outlet air temperature: 95° C., rotation speed: 20 rpm) to obtain a spray-dried powder. The spray-dried powder and microcrystalline cellulose were mixed in a ratio of 1:3. According to the dissolution rate test method described in Example 2 (1), the dissolution rate of 40 mg sample in pH 6.8 medium was detected. The results were shown in the table 24.

TABLE 23
Formulations with different proportion surfactant formulation

| Components | Formulation -44 | | Formulation -45 | | Formulation -46 | | Formulation -47 | | Formulation -48 | |
|---|---|---|---|---|---|---|---|---|---|---|
| Vilazodone hydrochloride | 10 | g | 10 | g | 10 | g | 10 | g | 10 | g |
| Carrier material | Copovidone | | | | | | | | | |
| Material weight | 60 | g | 60 | g | 60 | g | 60 | g | 60 | g |
| Surfactant | F68 | | | | | | | | | |
| Surfactant weight | 0.00 | g | 3.68 | g | 7.78 | g | 12.35 | g | 17.50 | g |

| Components | Formulation -49 | | Formulation -50 | | Formulation -51 |
|---|---|---|---|---|---|
| Vilazodone hydrochloride | 10 | g | 10 | g | 10 g |
| Carrier material | Povidone | | | | |
| Material weight | 50 | g | 50 | g | 50 g |
| Surfactant | Span-20 | | | | |
| Surfactant weight | 0.00 | g | 1.79 | g | 3.68 |

TABLE 24
Results of dissolution rate of formulations with different proportion
surfactant formulation in pH 6.8 media (Mean ± SD)

| Time | 5 min | 10 min | 15 min | 20 min | 30 min | 45 min |
|---|---|---|---|---|---|---|
| VIIBRYD ® | 3 ± 0.58 | 4 ± 0.00 | 4 ± 0.00 | 4 ± 0.00 | 4 ± 0.00 | 4 ± 0.58 |
| Formulation -44 | 48 ± 4.04 | 51 ± 3.61 | 53 ± 3.61 | 54 ± 3.21 | 54 ± 3.61 | 55 ± 3.51 |
| Formulation -45 | 65 ± 1.15 | 68 ± 2.00 | 69 ± 1.53 | 72 ± 0.58 | 71 ± 0.58 | 72 ± 2.00 |
| Formulation -46 | 64 ± 3.00 | 65 ± 3.21 | 66 ± 4.00 | 66 ± 2.52 | 66 ± 3.21 | 69 ± 3.06 |
| Formulation -47 | 62 ± 1.53 | 66 ± 3.21 | 66 ± 2.08 | 66 ± 1.53 | 66 ± 2.52 | 65 ± 3.46 |
| Formulation -48 | 60 ± 3.21 | 65 ± 0.58 | 65 ± 1.53 | 66 ± 1.15 | 68 ± 1.00 | 65 ± 2.31 |
| Formulation -49 | 69 ± 2.65 | 65 ± 10.58 | 54 ± 15.53 | 52 ± 14.19 | 52 ± 3.00 | 55 ± 1.53 |
| Formulation -50 | 75 ± 0.58 | 77 ± 1.00 | 79 ± 1.53 | 75 ± 2.65 | 76 ± 0.58 | 75 ± 0.58 |
| Formulation -51 | 69 ± 10.21 | 78 ± 4.92 | 76 ± 3.79 | 79 ± 4.93 | 81 ± 0.58 | 80 ± 1.00 |

The above results showed that the dissolution rate of the solid dispersion was significantly improved after the surfactant was added. When the proportion of the surfactant was within 20%, the dissolution rate was relatively stable (decreased within 50%), and it was significantly higher than that of the reference formulation VIIBRYD® (40 mg strength), which could significantly increase the oral bioavailability of vilazodone under fasting condition.

### Example 6 Formulation of Vilazodone Solid Dispersion by Hot Melt Extrusion Process

According to the formulations in Table 25, the carrier material, surfactant and vilazodone free alkali powder were put into a plastic bag and mixed evenly. The mixture was added to the Thermo twin-screw hot melt extruder (extrusion temperature was 180° C., the screw speed was 100 revolutions per minute) to extrude the mixture. which was pulverized after cooling to obtain the vilazodone solid dispersion. The above solid dispersion powder and microcrystalline cellulose were mixed uniformly in a ratio of 1:3. According to the dissolution test method described in Example 2 (1), the dissolution rate of a 10 mg sample in a pH 6.8 medium was detected. The results were shown in Table 26.

TABLE 25
Different material formulations

| Components | Formulation -52 | | Formulation -53 | | Formulation -54 | |
|---|---|---|---|---|---|---|
| Vilazodone | 10 | g | 10 | g | 10 | g |
| HPMCAS-MF | 60 | g | 45 | g | 30 | g |
| Soluplus | — | | 15 | g | 30 | g |
| Poloxamer 188 | 7.78 | g | 7.78 | g | 5 | g |

TABLE 26
Results of dissolution rate of different formulations in pH 6.8 media (Mean ± SD)

| Time | 5min | 10 min | 15 min | 20 min | 30 min | 45 min |
|---|---|---|---|---|---|---|
| VIIBRYD ® | N/A | 2 ± 0.6 | 4 ± 0.6 | 4 ± 0.6 | 5 ± 0 | 6 ± 0.6 |
| Formulation -52 | 23 ± 0.00 | 34 ± 0.58 | 41 ± 1.53 | 49 ± 1.00 | 57 ± 1.73 | 66 ± 2.00 |
| Formulation -53 | 15 ± 1.53 | 30 ± 1.15 | 41 ± 4.93 | 44 ± 3.61 | 56 ± 5.51 | 62 ± 1.73 |
| Formulation -54 | 9 ± 0.00 | 17 ± 0.58 | 22 ± 1.15 | 27 ± 0.58 | 31 ± 0.58 | 40 ± 1.73 |

The above results showed that the dissolution rate of vilazodone solid dispersion prepared by hot-melt extrusion process in simulated intestinal juice (pH 6.8 medium) was significantly higher than that of the reference formulation VIIBRYD® (10 mg strength), suggesting the vilazodone solid dispersion prepared by the hot-melt extrusion process could significantly increase the oral bioavailability of vilazodone under fasting condition.

### Example 7 Study of the Pharmacokinetic in Beagle Dogs

(1) Preparation of Vilazodone Tablets and Determination of Dissolution Rate

The spray-dried powder of formulation 33 was selected as the raw material, and the materials were weighed according to Table 27 and mixed uniformly; the total mixed materials were passed through a 40-mesh sieve and directly compressed to obtain the vilazodone hydrochloride tablets. According to method for determination of dissolution rate described in Example 2 (1), the dissolution rate of the self-developed tablets in different media were tested. The results were shown in Table 28.

(2) Pharmacokinetic Experiment in Beagle Dog

The vilazodone hydrochloride tablets (10 mg strength s) were used to study the pharmacokinetics in Beagle dog. Six male, healthy adult Beagle dogs with a weight range of 15-20 kg were randomly divided into 2 groups for a double-crossover and two-cycle test. The pharmacokinetic properties of the self-developed vilazodone tablets (10 mg) were investigated on fasting and feeding (high-fat, high-calorie food) conditions. After oral administration to animals, whole blood was collected at 0.25, 0.5, 1, 2, 4, 6, 8, 10, and 24 hours; the whole blood were placed in a centrifuge tube containing K2EDTA anticoagulant at 4° C. and centrifuged at 3500 rpm for 10 min. The upper plasma was collected and stored at −70° C. for testing.

The LC-MS-MS method was used to detect the content of vilazodone in the plasma. The plasma concentration-time curve is shown in FIG. 5; the pharmacokinetic parameters were calculated by using WinNonlin 6.3 software non-compartmental model method, and the food effect evaluation standard was calculated based on the Ratio value, Ratio=Fed/Fast*100%. The specific results were shown in Table 28 (The PK data in animals of the reference formulation VIIBRYD® refers to the patent application WO2018082557A1). For a single animal in a given test animal groups, the value of Cₘₐₓ and Ratio of AUC were calculated according to this formula. After the Ratio value of each animal was added, the sum was divided by the number of tested animals to obtain the average value. If the average Ratio was in the range of about 70% to about 143%, it could be determined that the dosage form had no food effect and was equivalent when taken with food or on an empty stomach. That is to say, whether the dosage form was taken with food or on an empty stomach, both had basically the same bioavailability and efficacy. (Note: Fast stands for fasting administration, Fed stands for feeding administration; Test is self-developed vilazodone tablets, and RLD is the reference formulation VIIBRYD®)

TABLE 27
vilazodone tablet formulation

| Components | Proportion (%) | Preparation process |
|---|---|---|
| Vilazodone hydrochloride | 5.00 | Spray drying |
| Copovidone | 30.00 | |
| Poloxamer 188 | 3.89 | |
| Microcrystalline cellulose | 50.11 | Direct mixing |
| Crospovidone | 10.00 | |
| Colloidal silicon dioxide | 0.50 | |
| Magnesium stearate | 0.50 | |
| Total | 100.00 | Tablet |

TABLE 28
Dissolution rate of self-developed tablets in different dissolution media (Mean ± SD)

| Dissolution | | | | | | |
|---|---|---|---|---|---|---|
| media | 5 min | 10 min | 15 min | 20 min | 30 min | 45 min |
| PH 3.1 | 41 ± 9.02 | 78 ± 12.74 | 92 ± 2.52 | 95 ± 1.53 | 96 ± 2.08 | 98 ± 2.52 |
| PH 6.8 | 41 ± 3.06 | 85 ± 4.58 | 90 ± 1.53 | 92 ± 1.15 | 94 ± 1.53 | 94 ± 1.00 |
| 0.1 MHCl | 65 ± 20.00 | 81 ± 18.52 | 87 ± 9.61 | 89 ± 7.55 | 91 ± 6.03 | 92 ± 4.16 |

TABLE 29
Pharmacokinetic data of vilazodone tablets under fasting
and feeding state (Mean ± SD, n = 6)

| | Tₘₐₓ | Cₘₐₓ | AUCₗₐₛₜ |
|---|---|---|---|
| Group | (h) | (ng/mL) | (h*ng/mL) |
| Test-Fed | 1.33 ± 0.58 | 56 ± 36 | 273 ± 170 |
| Test-Fast | 0.83 ± 0.29 | 62 ± 27 | 240 ± 120 |
| Test-(Fed/Fast) % | — | 90.32 | 113.75 |
| RLD-Fast | 1.50 ± 0.55 | 25 ± 19 | 109 ± 94 |
| RLD-Fed | 2.33 ± 1.86 | 77 ± 57 | 267 ± 188 |
| RLD-(Fed/Fast) % | — | 308.00 | 244.95 |

The results showed that the Cₘₐₓ and AUC of the self-developed vilazodone composition tablet in the fasting (Test-fast) and the fed (Test-fed) state were close, and the ratios were both in the range of 70% to 143%, considering that there was no food effect. It showed that the formulation prepared by the solid dispersion of vilazodone in this example could significantly improve the bioavailability of the fasting state, eliminate the influence of food, and make the drug can be taken in both the fasting and the fed state; but the ratio of Cmax and AUC of the commercial formulation VIIBRYD® (RLD, 10 mg) in the fasting and fed state were far less than the range of 700%˜143%, and there was a significant food effect. The above results suggested that the vilazodone solid dispersion preparation provided herein could significantly improve the bioavailability of vilazodone when taken on an empty stomach, eliminate the influence of food, and could be taken under both fasting and fed conditions, which significantly improved the patient's medication convenience and compliance, it was of great clinical significance for patients with severe depression who needed long-term medication and were often accompanied by loss of appetite and suicidal tendencies.

### Example 8 Accelerated Stability Investigation

The mixture prepared according to the proportion of formulation 28, formulation 33 and formulation 41 was spray-dried to obtain a spray-dried powder, which was sealed and packaged in double aluminum bags, and stored at 40° C. and 75% RH (humidity) for 2 months; after reaching the time point, samples were taken to determine the crystal form of each formulation vilazodone hydrochloride and the dissolution rate in the pH 6.8 medium in vitro. The XRD powder diffraction pattern of each formulation spray-dried powder samples after storage for 2 months is shown in FIG. 6, and the dissolution rate results are shown in Table 29; the results showed that after the vilazodone hydrochloride solid dispersion prepared by different formulations were stored under accelerated conditions for 2 months, they still existed in an amorphous form and the in vitro dissolution rate was stable, suggesting that the vilazodone solid dispersion provided herein had good stability.

TABLE 30
Dissolution rate results of spray-dried powders with different formulation in pH 6.8 media

| formulations | Time | | 5 min | 10 min | 15 min | 20 min | 30 min | 45 min |
|---|---|---|---|---|---|---|---|---|
| formulation 28 | 0 | day | 65 ± 1.15 | 73 ± 3.21 | 74 ± 0.00 | 75 ± 3.21 | 77 ± 2.08 | 77 ± 1.00 |
| | 2 | months | 69 ± 3.06 | 76 ± 1.00 | 74 ± 2.31 | 75 ± 3.00 | 78 ± 3.06 | 78 ± 0.58 |
| formulation 33 | 0 | day | 64 ± 3.00 | 65 ± 3.21 | 66 ± 4.00 | 66 ± 2.52 | 66 ± 3.21 | 69 ± 3.06 |
| | 2 | months | 63 ± 2.83 | 64 ± 1.41 | 65 ± 0.71 | 66 ± 1.41 | 68 ± 0.71 | 66 ± 2.12 |
| formulation 41 | 0 | day | 69 ± 1.73 | 74 ± 1.53 | 76 ± 1.15 | 76 ± 0.58 | 74 ± 1.15 | 74 ± 2.52 |
| | 2 | months | 76 ± 1.15 | 77 ± 1.00 | 79 ± 1.53 | 77 ± 1.00 | 77 ± 1.15 | 77 ± 4.04 |

The technical features of the above-mentioned embodiments can be combined arbitrarily. To make the description concise, all possible combinations of the technical features in the above-mentioned embodiments are not described; however, as long as there is no contradiction in the combination of these technical features, they should be considered to belong to the scope described in the specification of the present invention. The above embodiments only express a few implementation modes of the present invention, and the description is specific, but it cannot be understood as a limitation on the patent scope of the present invention. It should be pointed out that for those of ordinary skill in the art, without departing from the concept of the present invention, several modifications and improvements can be made, and these all fall within the protection scope of the present invention.

What is claimed is:

1. A vilazodone solid dispersion, comprising: vilazodone, a carrier material and a surfactant.

2. The vilazodone solid dispersion according to claim 1, wherein the carrier material is a water-soluble polymer carrier material, and the carrier material includes at least one selected from povidone, copovidone, and hydroxypropyl methylcellulose.

3. The vilazodone solid dispersion according to claim 2, wherein the weight ratio of the vilazodone to the water-soluble polymer carrier material is 1:4 to 1:7.

4. The vilazodone solid dispersion according to any one of claims 1-3, wherein the surfactant includes at least one selected from Span-20, polyoxyethylene 40 hydrogenated castor oil, caprylic acid capric acid polyethylene glycol glyceride, and poloxamer.

5. The vilazodone solid dispersion according to claim 1, wherein the weight ratio of the surfactant in the vilazodone solid dispersion is not more than 20%.

6. The vilazodone solid dispersion according to claim 1, wherein the weight ratio of the surfactant in the vilazodone solid dispersion is not more than 10%.

7. The vilazodone solid dispersion according to claim 1, wherein the carrier material is copovidone.

8. The vilazodone solid dispersion according to claim 7, wherein the weight ratio of the vilazodone to the copovidone is 1:4 to 1:7.

9. The vilazodone solid dispersion according to claim 8, wherein the surfactant is poloxamer 188.

10. The vilazodone solid dispersion according to claim 9, wherein the weight ratio of the poloxamer 188 in the vilazodone solid dispersion is not more than 20%.

11. The vilazodone solid dispersion according to claim 9, wherein the weight ratio of the poloxamer 188 in the vilazodone solid dispersion is not more than 10%.

12. The vilazodone solid dispersion according to claim 1, wherein the carrier material is povidone.

13. The vilazodone solid dispersion according to claim 12, wherein the weight ratio of the vilazodone to the povidone is 1:4 to 1:7.

14. The vilazodone solid dispersion according to claim 13, wherein the surfactant includes at least one selected from S pan-20 and poloxamer 188.

15. The vilazodone solid dispersion according to claim 14, wherein the surfactant is Span-20.

16. The vilazodone solid dispersion according to claim 15, wherein the weight ratio of the Span-20 in the vilazodone solid dispersion is not more than 10%.

17. The vilazodone solid dispersion according to claim 15, wherein the weight ratio of the Span-20 in the vilazodone solid dispersion is not more than 5%.

18. The vilazodone solid dispersion according to claim 1, wherein the carrier material is hydroxypropyl methylcellulose.

19. The vilazodone solid dispersion according to claim 18, wherein the weight ratio of the vilazodone to the hydroxypropyl methylcellulose is 1:4 to 1:7.

20. The vilazodone solid dispersion according to claim 19, wherein the surfactant includes at least one selected from caprylic acid capric acid polyethylene glycol glyceride and polyoxyethylene 40 hydrogenated castor oil.

21. The vilazodone solid dispersion according to claim 20, wherein the weight ratio of the surfactant in the vilazodone solid dispersion is not more than 10%.

22. A vilazodone solid dispersion, comprising vilazodone and a water-soluble polymer carrier material, wherein the water-soluble polymer carrier material is a mixed material, and the mixed material includes at least two selected from povidone, copovidone and hydroxypropyl methylcellulose.

23. The vilazodone solid dispersion according to claim 22, wherein the mixed material comprises copovidone and hydroxypropyl methylcellulose.

24. The vilazodone solid dispersion according to claim 22, wherein the mixed material comprises povidone and hydroxypropyl methylcellulose.

25. The vilazodone solid dispersion according to claim 22, wherein the weight ratio of the vilazodone to the mixed material is 1:4 to 1:7.

26. A method for preparing the vilazodone solid dispersion according to any one of claims 1-25, comprising the following steps: vilazodone and the carrier material weighed according to proportion are added to solvent and stirred to be dissolved, then spray-dried in a spray dryer, and the powder is collected to obtain the vilazodone solid dispersion.

27. The method according to claim 26, wherein the solvent is an acetone aqueous solution, and the volume ratio of acetone is greater than 60% and less than or equal to 70%.

28. Use of the vilazodone solid dispersion according to any one of claims 1-25 in the preparation of vilazodone formulation.

29. The use according to claim 28, wherein the dosage form of the vilazodone formulation includes granule, powder, dry suspension, tablet or capsule.

30. The use according to claim 29, wherein the tablet is a coated tablet.
